(19) United States
(12) Patent Application Publication
(10) Pub. No.: US 2017/0281857 A1
(43) Pub. Date: Oct. 5, 2017

(54) SYRINGE PUMP AND DRIVE HEAD ASSEMBLY THEREFOR

(71) Applicant: INNOVFUSION PTE LTD, Singapore (SG)

(72) Inventors: Ron WIGHT, Singapore (SG); Theodore TAN, Singapore (SG); Gabriel TAN, Singapore (SG)

(21) Appl. No.: 15/626,364

(22) Filed: Jun. 19, 2017

Related U.S. Application Data
(63) Continuation-in-part of application No. PCT/SG2015/050496, filed on Dec. 18, 2015.

(30) Foreign Application Priority Data
Dec. 19, 2014 (SG) 10201408525X

(51) Int. Cl. A61M 5/145

(52) U.S. Cl. CPC A61M 5/1458; A61M 5/1452; A61M 5/168

(57) ABSTRACT

A syringe pump usable with a syringe having a barrel and a plunger, the plunger having a plunger flange, comprises a barrel retaining mechanism for holding the barrel, and a drive mechanism having a drive head assembly for driving the plunger along its axis. The drive head assembly comprises a flange retaining mechanism for retaining the plunger flange to the drive head assembly, and a load measuring device for measuring compressive force on the plunger flange, the load measuring device being movable within the drive head assembly transverse to the axis. The syringe pump comprises a self-centering mechanism having a pushing component which is arranged to move the load measuring device within the drive head assembly until the pushing component abuts against the plunger flange, such that a center of the load measuring device is aligned with the axis.

### CROSS-REFERENCE TO RELATED APPLICATIONS

This application is a bypass continuation-in-part of PCT application PCT/SG2015/050496, filed 18 Dec. 2015, which, in turn, claims priority to Singapore patent application 10 2014 08525X, filed on 19 Dec. 2014. The content of each of these applications is incorporated by reference as if fully recited herein.

### TECHNICAL FIELD

An infusion pump is a medical device commonly used to infuse fluids or medicaments to a patient. In particular, an infusion syringe pump is a device which drives a syringe plunger of a syringe at a predetermined rate, thereby expelling fluid from the syringe and then on through a tube to the patient.

### BACKGROUND

Typically, syringe pumps comprise a motor that drives a screw or gear mechanism to move a pushing surface (drive head) at a controllable rate. The drive head is connected to a screw drive mechanism for connecting the linear motion of the screw drive mechanism to the syringe plunger in order to empty the syringe. The rate of movement of the pushing surface or drive head can be chosen in order to infuse fluid from the syringe at the desired rate of administration.

The syringe plunger has a plunger flange which is connected to the syringe piston by a syringe plunger stem. When a syringe is mounted in the syringe pump, and the drive mechanism is activated, the drive head pushes against the plunger flange to push the plunger stem (and therefore the piston) within the barrel to drive the fluid from the syringe.

A problem with known syringe pumps is that they are not easily able to accommodate differently sized syringes. In particular, if a syringe is used which has a different barrel diameter than that for which the syringe pump is designed, the drive head may not be centered along the axis of the barrel and the plunger, thus resulting in a cantilevered arrangement which causes a bending moment about the support structure. Further, if a load cell or other similar sensor is used to measure the compressive force applied to the plunger, the offset between the load cell and the axis will tend to attenuate the compressive force on the load cell, thus giving an inaccurate reading and reducing the sensitivity of the load cell. This can be a particularly serious safety concern, because accurate force readings are necessary to ensure that any build up of fluidic pressure beyond that expected can be detected early enough to take remedial action (e.g., by automatically stopping and/or withdrawing the drive head).

It would be desirable to provide a syringe pump which overcomes or alleviates one or more of the above problems, or which at least provides a useful alternative to known syringe pumps.

### SUMMARY OF INVENTION

In a first aspect, the present invention provides a syringe pump usable with a syringe having a barrel and a plunger, the plunger having a plunger flange, the syringe pump comprising:

a drive mechanism having a drive head assembly for driving the plunger along its axis, the drive head assembly comprising a flange retaining mechanism for retaining the plunger flange to the drive head assembly, and a load measuring device for measuring compressive force on the plunger flange, and;

a self-centering mechanism having a pushing component which is arranged to move the load measuring device within the drive head assembly until the pushing component abuts against the plunger flange or a datum that corresponds to the size of the plunger flange, such that a center of the load measuring device is aligned with the axis.

In a second aspect, the invention provides syringe pump usable with a syringe having a barrel and a plunger, the plunger having a plunger flange, the syringe pump comprising: a housing arranged to receive the syringe, the housing having a drive mechanism having a drive head assembly for driving the plunger along its axis; the drive head assembly including a load measuring device for measuring compressive force on the plunger flange and a push button clamp arranged to clamp the plunger flange against the drive head assembly.

The pushing component may be located on the drive head assembly.

In some embodiments, the load measuring device is coupled to a transversely movable element having an engagement portion, and the pushing component has at least a portion which is arranged to contact the engagement portion to move the transversely movable element.

The flange retaining mechanism may comprise at least one rotationally mounted arm arranged to engage with the plunger flange. In some embodiments, the flange retaining mechanism comprises a pair of rotationally mounted arms. The pair of arms may be mounted symmetrically about an axis centered on the load measuring device.

In some embodiments, the pushing component is part of the flange retaining mechanism.

In some embodiments, at least one of the rotationally mounted arms is arranged to contact the engagement portion.

In some embodiments, a load transfer element may project from the transversely movable element.

In some embodiments, the drive mechanism comprises a pair of lead screws disposed within respective tie bars. The tie bars may be aligned with mounting points of the rotationally mounted arms.

In a third aspect, the present invention provides a drive head assembly for a syringe pump usable with a syringe having a barrel and a plunger, the plunger having a plunger flange, the drive head assembly being configured to drive the plunger along its axis when loaded in the syringe pump, the drive head assembly comprising:

a flange retaining mechanism for retaining the plunger flange to the drive head assembly, and a load measuring device for measuring compressive force on the plunger flange, the load measuring device being movable within the drive head assembly transverse to the axis;

wherein the load measuring device is arranged to receive a pushing force from a pushing component of a self-centering mechanism such that the load measuring device is moved within the drive head assembly until the pushing component abuts against the plunger flange or a datum that corresponds to the size of the plunger flange, such that a center of the load measuring device is aligned with the axis.

In a fourth aspect, the present invention provides a method of loading a syringe in a syringe pump according to the first aspect of the invention, the syringe having a barrel with a barrel flange, and a plunger arranged to move within the barrel and having a plunger flange, the method comprising:

placing the barrel in a cradle of the syringe pump;

activating the flange retaining mechanism to retain the plunger flange; then,

activating the drive mechanism to push the plunger flange such that the barrel flange abuts against an engagement surface of the syringe pump.

### BRIEF DESCRIPTION OF THE DRAWINGS

Embodiments of the invention will now be described, by way of non-limiting example only, with reference to the accompanying drawings in which:

FIG. 1 is a first perspective view of a syringe pump according to one embodiment of the present invention;

FIG. 2 shows the same view as FIG. 1, but with a syringe loaded in the syringe pump;

FIG. 3 is a second perspective view of the syringe pump of FIG. 1 and FIG. 2;

FIG. 4 is the same view as FIG. 3, but showing a syringe loaded in the syringe pump;

FIG. 5 is an exploded view of a drive head assembly of the syringe pump of FIG. 1 to FIG. 4;

FIG. 6 to FIG. 8 show a close-up view of the drive head assembly during loading of a syringe;

FIG. 9(a) is a plan view of the drive head assembly in a first configuration;

FIG. 9(b) is a cross-section through the line A-A of FIG. 9(a);

FIG. 10(a) is a plan view of the drive head assembly in a second configuration; and

FIG. 10(b) is a cross-section through the line B-B of FIG. 10(a);

FIG. 11 is an isometric view of a syringe pump according to one embodiment of the present invention;

FIG. 12 is a side elevation view of a syringe according to one embodiment of the present invention;

FIGS. 13A to 13C are various views of a syringe pump according to a further embodiment of the present invention;

FIGS. 14A to 14C are various views of the syringe pump as shown in FIGS. 13A to 13C, and;

FIGS. 15A to 15B are various views of a syringe pump according to a further embodiment of the present invention.

### DETAILED DESCRIPTION OF EMBODIMENTS

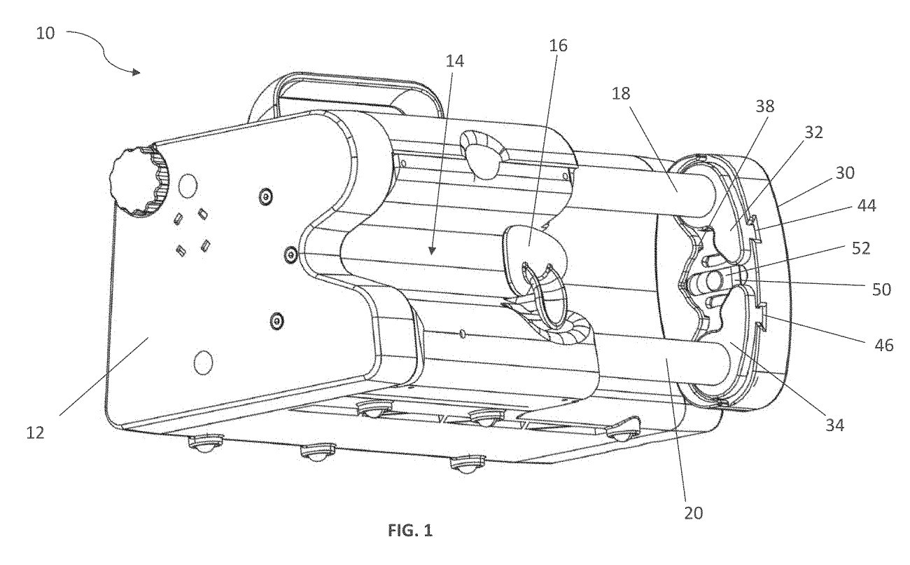

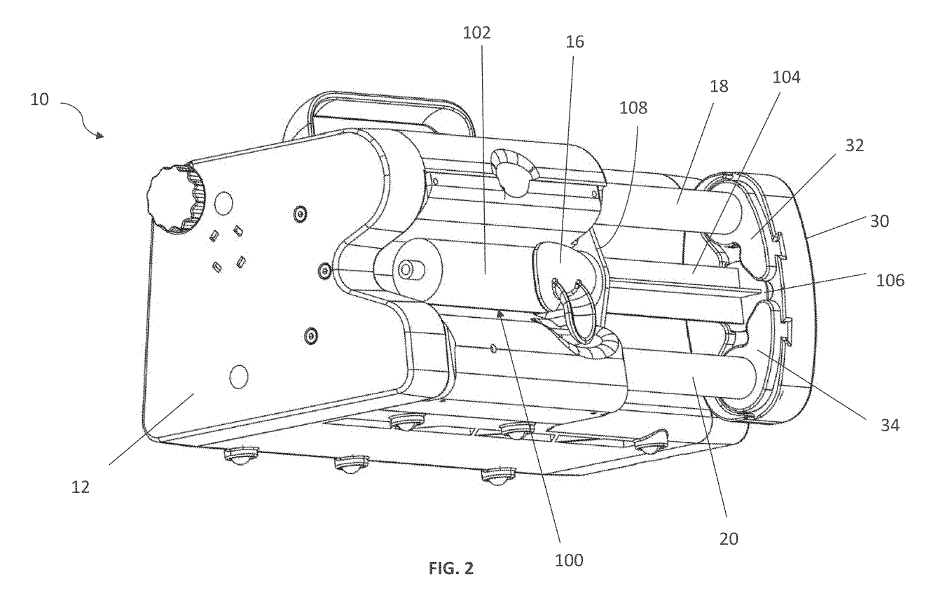

Referring to FIG. 1 and FIG. 2, there is shown an embodiment of a syringe pump 10 having a body 12 with a syringe cradle 14 which receives the barrel 102 of a syringe 100. The barrel 102 is retained within the syringe cradle 14 by a syringe barrel clamp or clip 16 which may be spring-loaded.

A linear potentiometer (not shown) may be mounted to the syringe barrel clamp 16 to automatically detect the external diameter of the barrel of a mounted syringe to identify the capacity (and possibly the brand) of the syringe, for example using a lookup table which is stored in a memory of a microcontroller of the syringe pump. The correct identification and verification of the make and model of the mounted syringe is important as it may affect the titration accuracy of the pump.

The syringe pump 10 has a drive mechanism comprising a first tie bar 18 and a second tie bar 20, respective ends of which are coupled to a drive head assembly 30. The tie bars 18, 20 are positioned symmetrically about a center of the drive head assembly 30. Each tie bar 18, 20 houses part of the length of a lead screw. Each lead screw is coupled at one end to a stepper motor (not shown) which causes the lead screws to rotate, thereby causing the drive head assembly 30 to move linearly. When a syringe 100 is engaged within the syringe pump 10, as shown in FIG. 2, a contact surface of the drive head assembly 30 pushes against the plunger flange 106 to drive the plunger 104 within the barrel 102 of the syringe 100.

Movement of the drive head assembly is controlled by a microcontroller of standard type (not shown) located within the housing 12 of the syringe pump 10. The microcontroller is coupled to, and in control of, various components of the pump 10, including motors and sensors (such as a linear potentiometer for tracking the position of the syringe plunger, an optical sensor for detecting the home position of the syringe, a switch 24 for detecting that the syringe barrel is correctly positioned, etc.). Further, the microcontroller may have one or more interfaces for connecting the pump to other devices, such as a (wired or wireless) network interface or a serial port, to allow the microcontroller to receive data from and to transmit data to such other devices (such as laptop or desktop computer systems, or mobile computing devices such as smartphones or tablets).

When secured within syringe pump 10, the syringe 100 is disposed with an edge portion of the plunger flange 106 nested in a semi-circular channel 38 (FIG. 1), and with fingers of a pair of rotatable arms 32 and 34 exerting a force on an opposite edge portion of the plunger flange 106, in a direction which is transverse to the longitudinal axis of the plunger barrel 102 and of the plunger 104. The plunger flange 106 is thus securely gripped between the arms 32, 34 and the semi-circular channel 38.

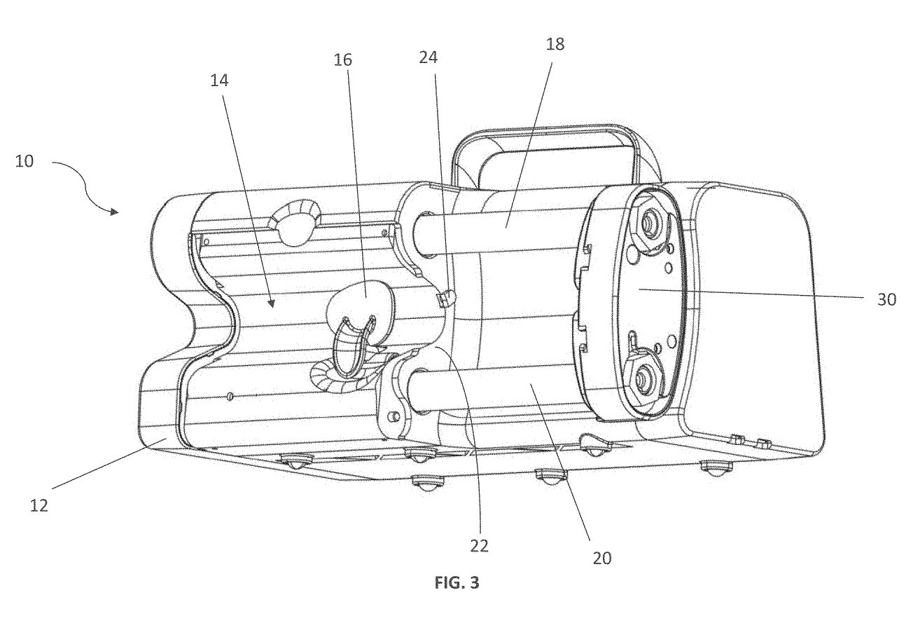

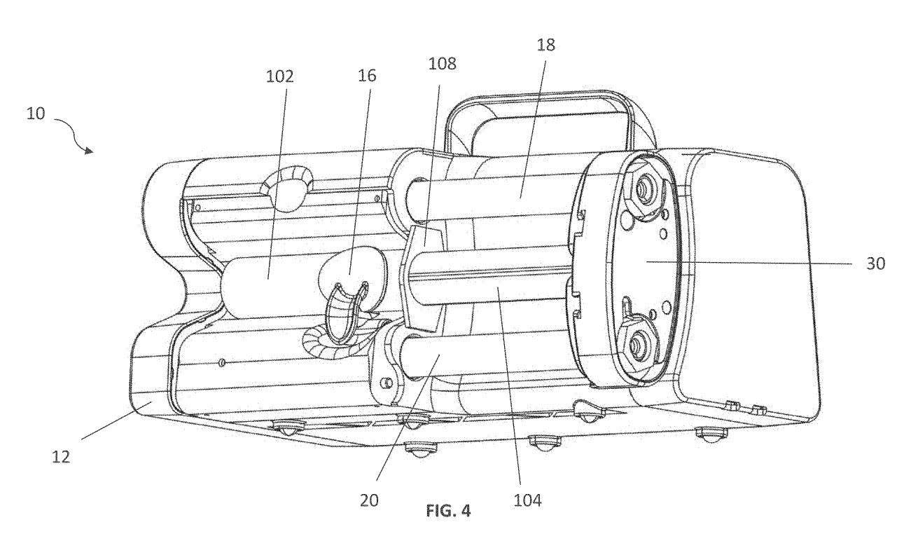

Turning to FIG. 3 and FIG. 4, the housing 12 has an engagement surface 22 against which the flange 108 of barrel 102 bears when the drive head assembly 30 urges the plunger 104. Once the syringe 100 is positioned, the pump 10 “primes” the syringe position by advancing the drive head 30, pushing the syringe plunger 104 and syringe barrel 102 in tandem until the syringe body 102 has advanced sufficiently to be flush with the engagement surface 22 and the flange 108 on the syringe barrel triggers a spring-loaded switch 24 to indicate its position. Since the syringe barrel flange 108 acts against the housing 12, any further advancement of the drive head 30 will lead to the syringe plunger 104 compressing against the stationary syringe barrel 102, dispensing the fluid in the syringe 100.

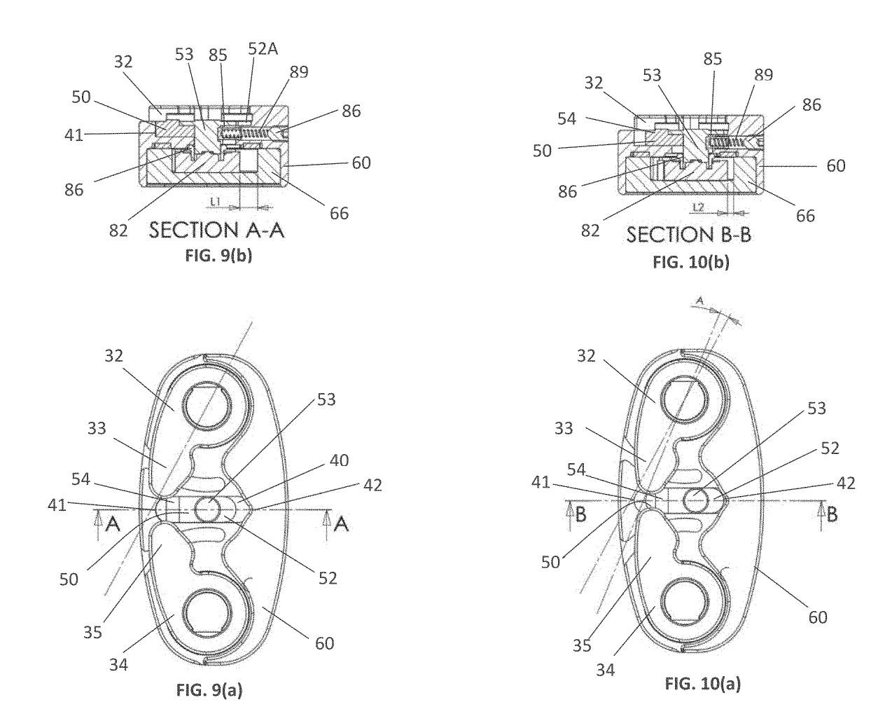

As shown in FIG. 9(b), the drive head assembly 30 comprises a load measuring device in the form of a compression load cell 82. The load cell 82 is coupled to a load transfer element 52 which has a contact element 53 projecting slightly from its surface. The contact element 53 presses against the syringe flange 106 when the drive mechanism of the syringe pump 10 is active, such that the load transfer element transmits a compressive force to the load cell 82. The load cell 82 is coupled to the microcontroller (not shown), which can infer the fluid pressure in the syringe barrel 102 based on the force measurement. The drive head assembly 30 also comprises a self-centering mechanism for aligning the axis of the plunger 104 with the center of the contact element 53, thereby ensuring that an accurate reading of the in-line fluidic pressure can be obtained.

The arms 32 and 34 of the drive head assembly 30 are mounted for rotation about the respective tie bars 18 and 20, and are mechanically linked by a gear train within the drive head assembly 30. The arms 32 and 34 need not rotate about the tie bars 18 and 20 so long as they can engage with the centering wedge 50 of the self-centering mechanism described below.

Each arm 32, 34 has a respective lower T-section portion 32A, 34A which moves within a corresponding arcuate groove 44, 46 on the drive head body 60. The T-section portions 32A, 34A, in conjunction with the respective grooves 44 and 46 in which they are disposed when the arms 32, 34 grip the plunger flange 106, and in conjunction with the channel 38 in which the flange 106 is seated, ensure that if the drive head assembly 30 is reversed, mechanical resistance from the plunger flange 106 during retraction does not displace the arms 32, 34. This allows the plunger 104 to be retracted from the barrel 102 using the drive head assembly 30.

As shown in the exploded view of FIG. 5, the gear train comprises a first spur gear 70 associated with the first arm 18 and a second spur gear 72 associated with the second arm 20. The first and second spur gears 70 and 72 are coupled to a large idler gear 74, a pair of smaller idler gears 97, and a drive gear 98. The drive gear 98 may be coupled to a stepper motor (not shown) in the drive head assembly 30. The coupling of the arms 32 and 34 by the gear train means that the clockwise rotation of one drive arm 32 will result in equal but anticlockwise rotation of the other drive arm 34. The paired inward rotation of the drive arms encircles and retains the flange 106 of syringe plunger 104 against the channel 38 of the drive head assembly 30. The symmetry in rotation of the drive arms also ensures that the circular plunger flange 106 is properly centered.

In some embodiments, the arms 32 and 34 can be motorized with a stepper motor and rotary encoder. This means that the diameter of the plunger flange can be measured when the arms 32 and 34 have gripped the flange, providing a measurement of the syringe size. The measurement may be used to determine the syringe type from a lookup table, and/or to confirm a previous syringe type identification based on a measurement of the barrel external diameter, for example.

When the pump 10 is active and the drive head 30 advances, the compressive force from the syringe plunger 104 translates through the load transfer button 52 and is measured by the compression load cell 82 as described above.

The self-centering mechanism of the drive head assembly 30 comprises a centering wedge 50, the load transfer button 52, a location ring 86 and compression load cell sensor 82. Referring to FIG. 5, the load transfer button 52 comprises a plate-like portion 52A from which the contact element 53 projects. Contact element 53 has a first portion projecting above the plate 52A (i.e., on the side of the plate facing the plunger flange 106 when the pump 10 is in use) and a second portion, which is longer than the first portion, projecting below the plate 52A. The second portion projects into the housing of the drive head assembly 30 and in particular, abuts at part of its circumference against an arcuate groove 55 of the centering wedge 50, and at its bottom against the load cell 82. The location ring is seated inside a centrally located circular groove of the load cell 82 and also receives the second projecting portion of the contact element 53, such that the contact element is correctly centered on the load cell 82, to give the most accurate reading possible.

It will be appreciated that in some embodiments, separate parts of the self-centering mechanism may be formed as a single integral component. For example, in some embodiments the centering wedge 50 and load transfer button may be a single component.

The centering wedge 50, the load transfer button 52, location ring 86 and the compression load cell sensor 82 are thus coupled together, and are constrained to move linearly within a stadium (or discorectangle) shaped groove 40 in the drive head housing 60. The groove 40 has a first end 41 having a shape complementary to an end of the centering wedge 50, and a second end 42 having a shape complementary to an end of the plate 52A of load transfer button 52 (FIG. 9(a)). The groove 40 also has a width which is slightly larger than the width of the plate 52A to ensure a close running fit between the components and the bulk of the drive head. The self-centering assembly therefore has a range of motion between a first position, as seen in FIG. 9(a) and FIG. 9(b), at which the centering wedge 50 abuts against the first end 41; and a second position, as seen in FIG. 10(a) and FIG. 10(b), at which the plate 52A abuts against the second end 42 of the groove 40. This range of motion determines the range of plunger flange 106 diameters which can be accommodated by the self-centering mechanism, as will later be described. Accordingly, the skilled person will appreciate that the dimensions of the self-centering mechanism, and of the groove 40, may be chosen to accommodate any desired range of plunger flange diameters, for example according to the typical range of flange diameters of syringes used with infusion syringe pumps.

The self-centering assembly enables the load transfer button 52 and the compression load cell sensor 82 to be repositioned concentrically with the center of the syringe plunger 104. The linear motion of the self-centering assembly is restricted by a detent bar 85 which is biased towards the first end 41 by a spring 89, and a set screw 96, which holds the detent bar 85 in place in the drive head housing 60 and which may be used to adjust the tension of the spring 89.

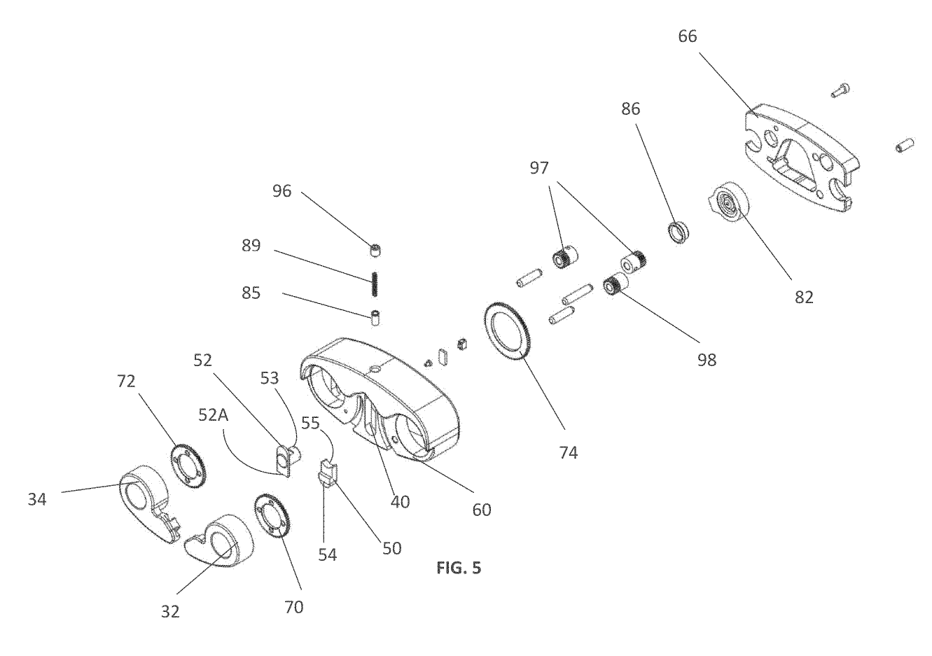

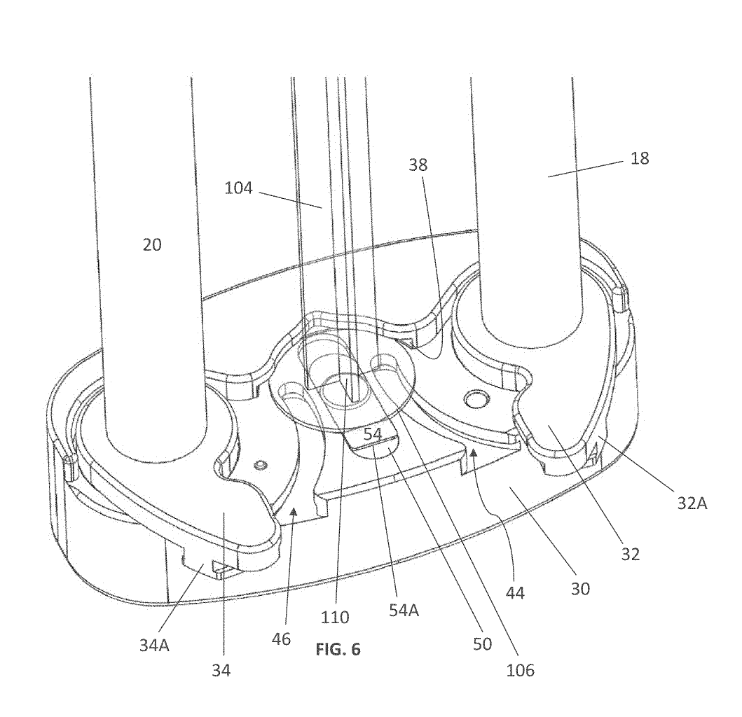

The operation of the self-centering mechanism may be understood from the following example. In order to load a syringe 100, a user places the syringe in the cradle 14 and positions the flange 106 in the channel 38, continuing to hold the barrel 102 in the cradle 14. The clamp 16 is then used to secure the syringe barrel 102 in the cradle 14. As shown in the close-up view of FIG. 6, the plunger 104 remains unsecured at this point. In addition, the center of the contact element 53 of the load transfer button 52 is offset from the axis of the plunger 104, which meets the plunger flange 106 at point 110. As can be seen in FIG. 6, a forward edge 54A of a stop 54 of the centering wedge 50 extends beyond the outwardly facing edge of the plunger flange 106.

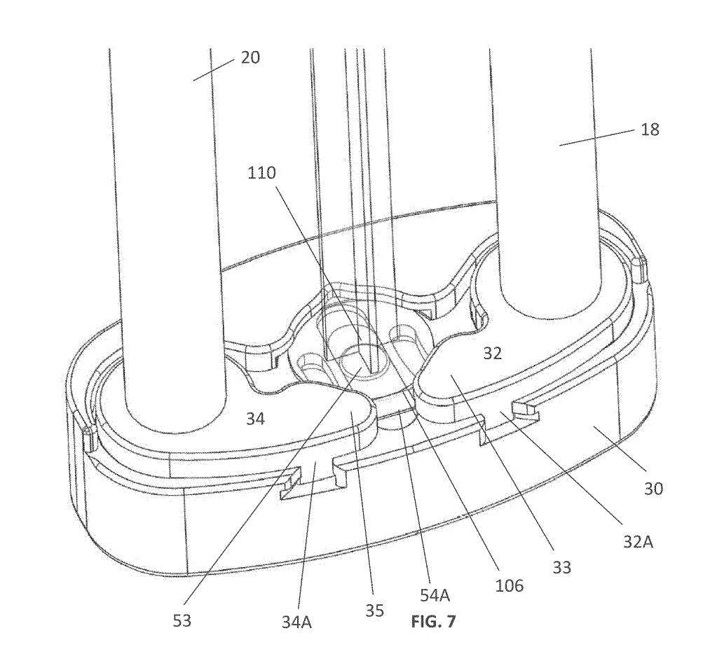

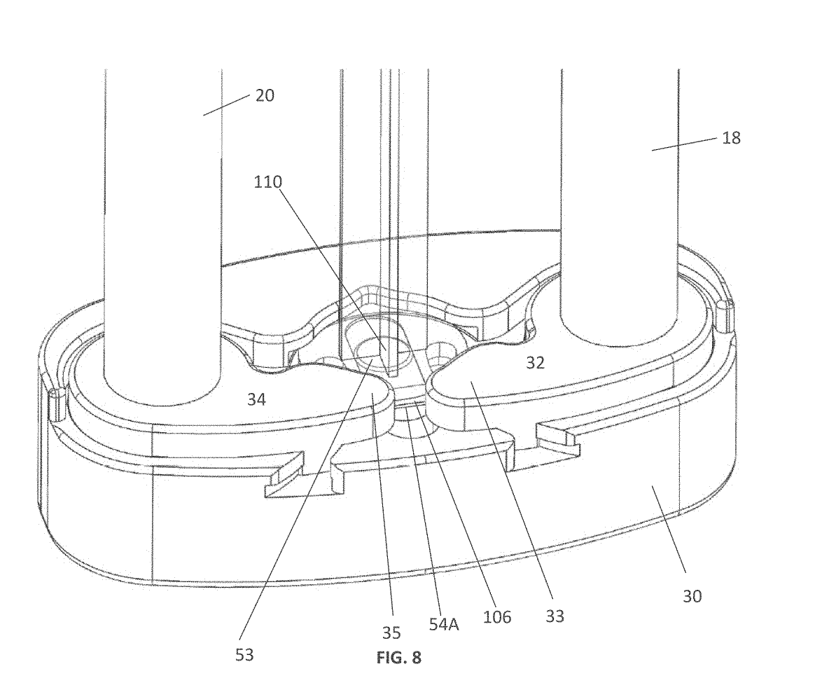

In order to secure the syringe 100 firmly in place the rotating arms 32 and 34 are rotated inwardly about the respective tie bars 18, 20 towards the edge of the plunger flange 106. Since the forward edge 54A of the stop 54 protrudes beyond the edge of the plunger flange 106, the fingers 33, 35 of arms 32, 34 meet the forward edge 54A first, as seen in FIG. 7. As the arms 32, 34 continue to rotate inwardly they drive the centering wedge 50 and the components to which it is coupled (the load transfer button 52, load cell 82 and locating ring 86) to linearly translate along the groove 40, acting against the spring 89 biasing the detent bar 85 (see FIGS. 10(a) and 10(b)). The arms 32, 34 continue to rotate until the fingers 33, 35 engage with the edge of the plunger flange 106, at which point the plunger 104 is secured and centered on the load transfer element 53 as shown in FIG. 8.

Advantageously, in the presently described embodiments where the flange 106 is seated in channel 38, the plunger flange 106 is engaged first followed by the engagement of barrel flange 108 on surface 22, through the advancement of drive head 30 which pushes the whole syringe 100 forward by applying a pushing force to the plunger flange 106. Therefore, the syringe is pushed forward instead of remaining stationary during the engagement of the flanges 106, 108. This permits the syringe 100 to be primed and ready to dispense fluid.

It will be appreciated that if the plunger flange 106 is of sufficiently large diameter then the forward edge 54A will be flush with the flange 106 edge and the axis of the plunger 104 will already be aligned with the center of the contact element 53, in which case no alignment correction would take place during securing of the plunger 104 by the arms 32, 34. If the diameter is too large then the forward edge 54A of the stop 54 will be behind the flange 106 edge such that the center of contact element 53 is misaligned and the arms 32, 34 cannot move the stop 54 to perform the alignment correction. As such, the plunger dimensions for which self-centering will work are constrained by the dimensions of the components of the self-centering mechanism.

The objective of the compression load cell 82 is to detect the amount of force that the drive head 30 is applying to the syringe plunger 104 when the drive head 30 advances. This force correlates with the in-line fluidic pressure within the syringe 100. In cases where the fluidic path is blocked, for example due to kinks in the tubing, a dislodged catheter or debris, continual advancement of the drive head 30 will build up the fluidic pressure. When sufficiently high, the pressure, when relieved, can potentially cause a sudden high volume burst of fluid to the patient or induce rupture of the tubing or catheter. Hence, an accurate reading of the in-line fluidic pressure, through the compressive force on the load cell 82, is a critical safety factor.

Whenever a load cell is not directly centered on the direction of the force that is to be measured, the force has to be translated through a load-carrying member that is axially offset to the direction of force. The offset induces a “cantilever beam” bending effect on the load-carrying member that attenuates the amount of compressive force on the load cell on the end of the offset member. This effectively means that a load cell 82 when not centered on the direction of force will have reduced sensitivity which is proportional to the length of the offset. Advantageously, therefore, the self-centering mechanism provided in the drive head assembly 30 minimizes the amount of offset, thus enabling maximum sensitivity of the load cell 82 to the compressive force on the syringe plunger 104.

Although particular embodiments of the invention have been described, it will be appreciated by the skilled reader that many variations and modifications are possible, while still falling within the scope of the invention. For example, although the plunger flange retaining mechanism comprises a pair of rotational arms 32 and 34, in some embodiments only a single rotating arm may be used to grip the plunger flange 106. Alternative means of retaining the plunger flange are also possible, for example a vice clamp mechanism having a linearly moving jaw which advances towards the flange 106 so that the flange 106 is retained between the channel 38 and the moving jaw. In this case, part of the moving jaw may engage with the stop member 54 of the centering wedge 50 in order to align the center of the load cell button 53 (and thus of the load cell 82) with the plunger axis 110. In some examples, centering of the load cell button 53 may be achieved by a separate pushing component which is not part of the flange retaining mechanism, such as an arm which engages with the stop member 54 to advance the self-centering mechanism until the arm abuts against the edge of the flange 106.

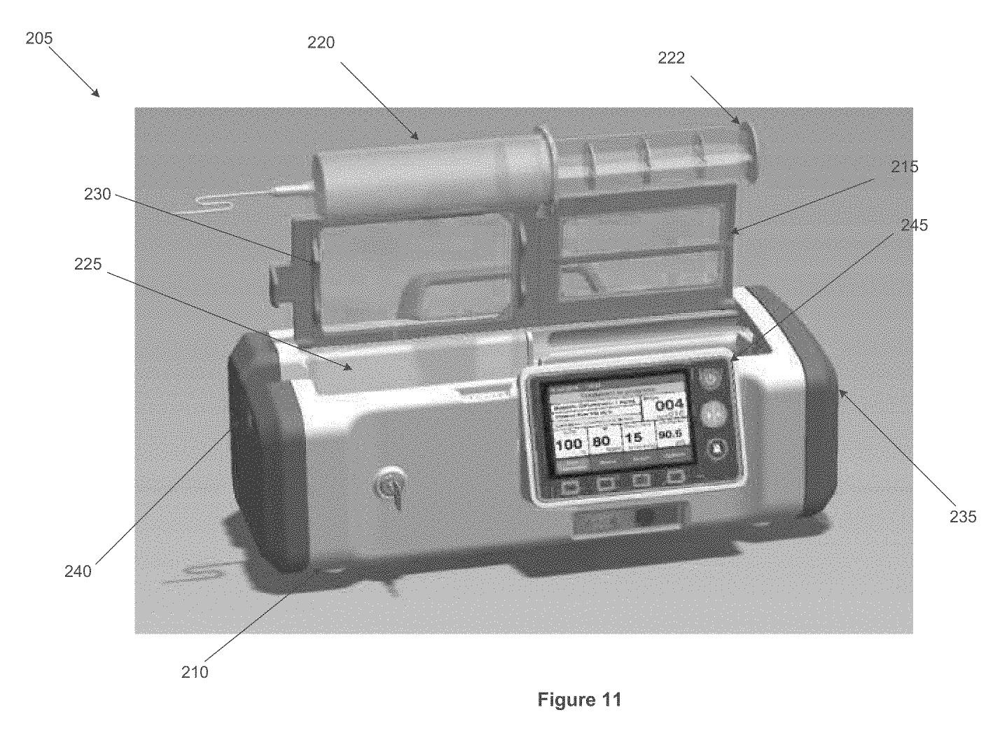

FIG. 11 shows a syringe pump 205 according to one embodiment of the present invention. The syringe pump 205 comprises a housing 210 having a recess 225 for receiving a syringe 220. The syringe fits within the recess 225 so that the plunger flange 222 sits up against the drive head assembly 235 and the syringe nozzle projects through an aperture 40 so as to be connectable to a tube for delivering a therapeutic house within the syringe 220.

Thus, the syringe pump 205 acts to automatically deliver the therapeutic according to a control system either resident within the syringe pump or externally connected to a processor (not shown).

Once the syringe 220 is within the recess 225 the housing is closed through closing the security door 215 such that barrel brace 230 fixed to the security door engage the barrel, holding the syringe firmly in place so as to prevent relative movement of the syringe during operation under the load from the drive head assembly 235.

A screen 245 is also provided on the housing 210 so as to provide data on the progress of the injection of the therapeutic. The screen 245 may also be a touch screen so as to feed control parameters into the syringe pump 205 should the syringe pump 205 be a self-contained unit having the control system resident therein.

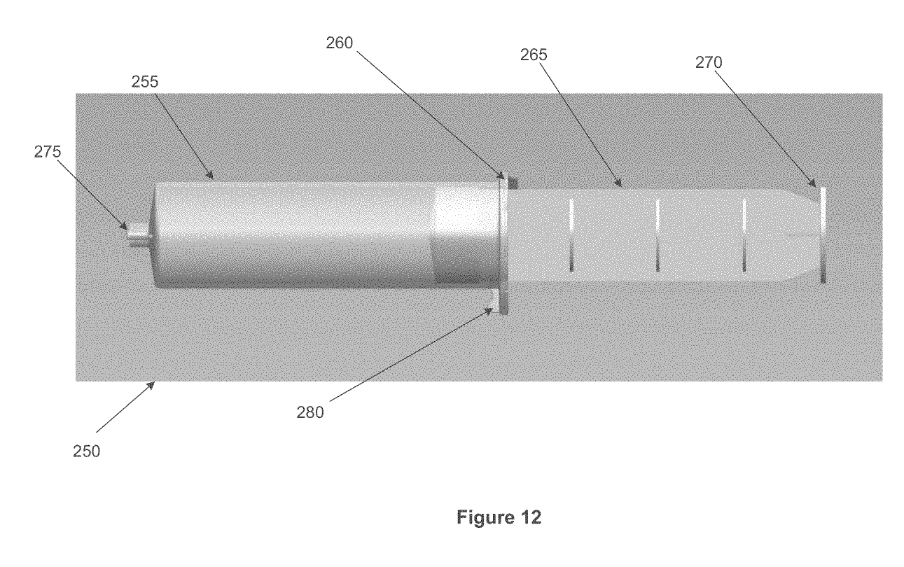

FIG. 12 shows a syringe usable with the syringe pump of FIG. 11. Here the syringe 250 includes a barrel 255 and barrel flange 260. A plunger, for applying pressure to the therapeutic within the barrel, includes a plunger flange 270 for receiving an applied force to exert the aforementioned pressure. The nozzle 275 acts as the vent through which the therapeutic can then be directed. The embodiment of the syringe 250 shown in FIG. 12 is further adapted to include a safety projection 280. For this adapted syringe 250 the safety projection is arranged to fit within a safety aperture within the recess of the syringe pump. The safety projection and safety aperture ensure the safe engagement of the syringe within the recess. In one embodiment the syringe pump according to the present invention may be prevented from commencing operation until the safety projection has been received within the safety aperture. This ensures the drive head assembly is correctly aligned with the longitudinal axis of the syringe. As will be explained later, in order to provide a correct feedback as to the volume of therapeutic being injected, an accurate measure of the applied load from the drive head assembly is required. If the syringe is misaligned within the syringe pump, an incorrect result may be recorded and therefore an inaccurate measure of the injected therapeutic registered. It follows that such an outcome could be catastrophic to the intended use of the syringe pump. Accordingly, the safety projection being mounted within the safety aperture represents a critical safety feature of the syringe pump according to this embodiment of the present invention.

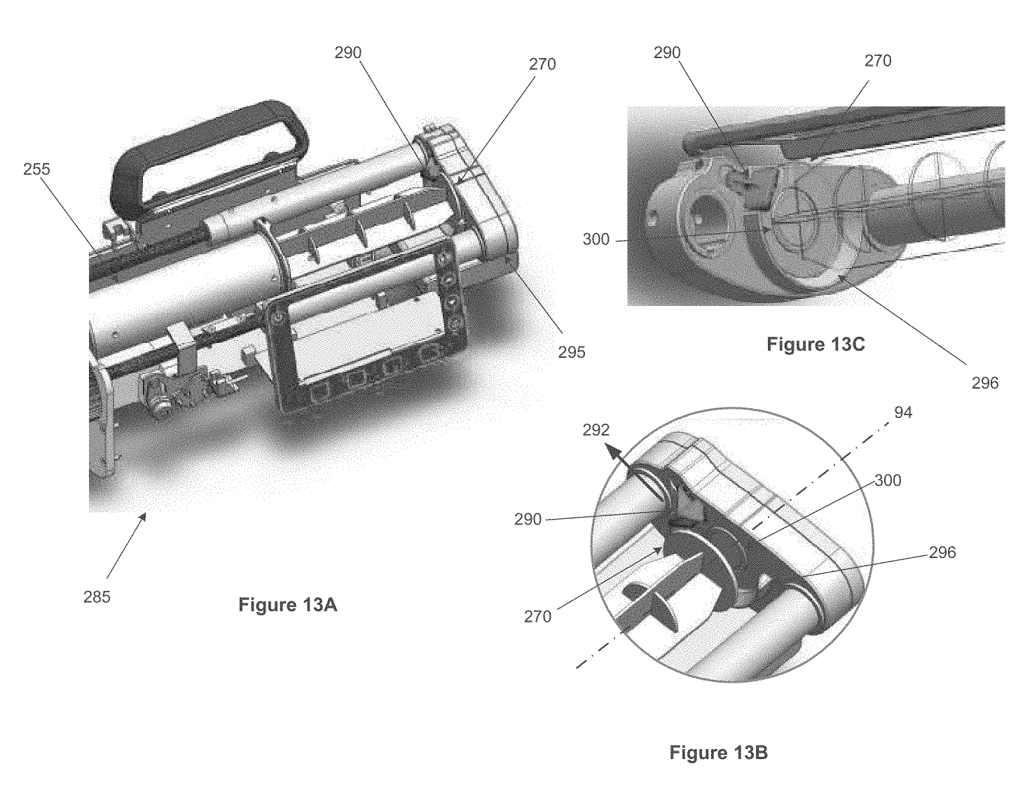

FIG. 13A shows a view where the barrel 255 has been placed within the recess and the plunger flange is held in place. The mechanism by which the plunger flange is held in place is a push button clamp 290 which, as shown in the details view of FIG. 13B, is biased away from a longitudinal axis 294 of the syringe. The intention is for the push button clamp to clamp the plunger flange in position and hold it against a flange bracket 296. The push button clamp 290 and flange bracket 296 are positioned to align the longitudinal axis 294 of the syringe so as to be centralized on a load cell 300 within the drive head assembly 295. Thus, the push button clamp not only acts as a means of holding the syringe in place but further acts as a locator to ensure the correct alignment of the drive head assembly 295 and the load cell 300 placed within it. FIG. 13C shows the ideal alignment with the push button clamp 90 holding the plunger flange 270 directly in alignment with the load cell 300.

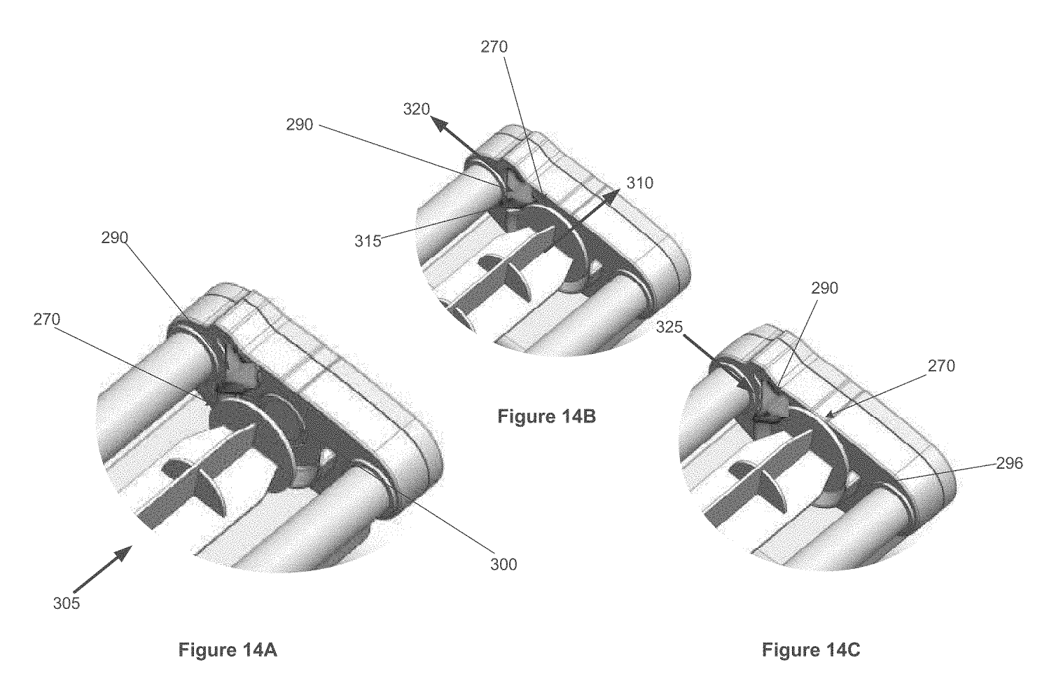

The process of fixing the plunger flange can be seen in FIGS. 14A to 14C. The push button clamp 290 and flange bracket 296 define a bay into which the plunger flange 270 is arranged to fit. The syringe is inserted 305 until it contacts the inclined face 315 of the push button clamp 290. On pushing against the inclined surface 315 the push button clamp is pushed outwards 320 against the spring loaded mechanism allowing passage 310 of the plunger flange 270 into the bay residing in the drive head assembly.

On full insertion into the bay, the plunger clamp 270 fits into an indentation in the push button clamp 290 allowing the push button clamp 290 to spring back 325 into position. The resilient bias of the spring loaded push button clamp 290 locates the plunger flange 270 to fit centrally within the bay by bearing against the bracket 296. Thus the push button clamp ensures the central location of the plunger flange 270 and ensures the longitudinal axis of the syringe is aligned with the load cell 300 within the drive head assembly.

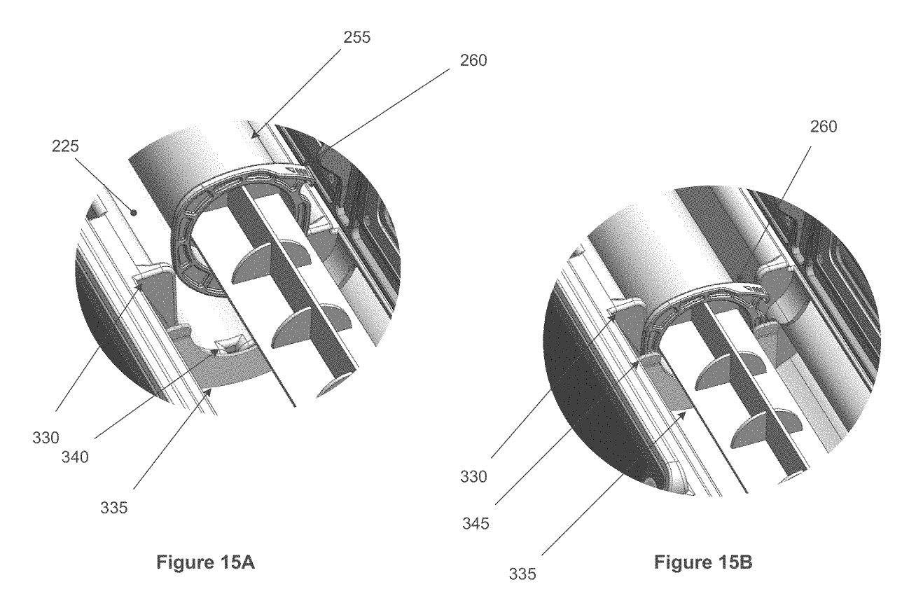

A feature of the syringe pump according to one embodiment of the present invention is the means by which the barrel flange 260 is secured so as to provide a reaction force against the drive head assembly depressing the plunger. FIGS. 15A and 15B show how the syringe fits within the recess 225. Here the barrel flange 260 slides between a pair of flange brackets 330, 335. The flange brackets 330, 335 are spaced so as to receive the barrel flange 260 there between, snugly so as to easily slide into place but not move along the longitudinal axis relative to the flange brackets. The flange bracket 235 which is positioned intermediate the barrel flange 260 and the plunger flange 300 is slightly flared 345 to facilitate the receiving of the barrel flange 260 between the flange brackets. The recess 225 further includes a projection aperture 340 for receiving the safety projection 280 as shown in FIG. 12. It will be appreciated that the use of the flange brackets allows a wide range of standard syringes to be used. In a further embodiment, an additional safety feature ensuring the proper alignment of the syringe is possible. When using a syringe according to one embodiment of the present invention, there will be a safety projection 280 insertable into a safety aperture 340. On insertion, the system is triggered to permit activation, but prevented from activation unless the safety projection is inserted within the safety aperture.

A syringe pump according to an alternative embodiment, may use a standard syringe, whereby the safety feature operated by the safety projection and safety recess is selectively disengaged.

It follows that a syringe pump according to a further embodiment of the present invention may omit the safety aperture 340 so as to permit the use of conventional syringes.

FIG. 15B shows the syringe fully inserted within the recess 225 such that the barrel flange 260 is securely positioned between the flange brackets 330, 335. Referring to FIG. 11 the security door 215 can be closed so as to place the barrel brace 230 over the barrel and further secure the syringe within the recess 225. By connecting the nozzle of the syringe to a tube, the syringe pump is then ready for operation.

It will be noted from the foregoing, that key features of the embodiments may be interchangeable between the syringe pumps as described. For instance, the push button clamp and fixed bracket of FIGS. 13A to 13C may be usable in a syringe pump that also contains the movable load measuring device of FIGS. 9(b) and 10(b). The pair of flange brackets shown in FIGS. 15A and 15B may be usable with the syringe barrel clamp shown in FIG. 2. To this end, the specification describes a range of features that may be incorporated, combined and exchanged to form different embodiments to those specifically grouped, whilst still falling within the scope of the invention.

What is claimed is:

1. A syringe pump usable with a syringe having a barrel and a plunger, the plunger having a plunger flange, the syringe pump comprising:
a drive mechanism having a drive head assembly for driving the plunger along its axis, the drive head assembly comprising a flange retaining mechanism for retaining the plunger flange to the drive head assembly, and a load measuring device for measuring compressive force on the plunger flange, and
a pushing component which is arranged to advance towards the load measuring device and to move the load measuring device within the drive head assembly until the pushing component abuts against the plunger flange or a datum that corresponds to the size of the plunger flange, such that a center of the load measuring device is aligned with the axis.

2. A syringe pump according to claim 1, further including a barrel retaining mechanism arranged to hold the barrel.

3. A syringe pump according to claim 1, wherein the load measuring device being movable within the drive head assembly transverse to the axis.

4. A syringe pump according to claim 1, wherein the pushing component is located on the drive head assembly.

5. A syringe pump according to claim 3, wherein the load measuring device is coupled to a transversely movable element having an engagement portion, and wherein the pushing component has at least a portion which is arranged to contact the engagement portion to move the transversely movable element.

6. A syringe pump according to claim 1, wherein the flange retaining mechanism comprises at least one rotationally mounted arm arranged to engage with the plunger flange.

7. A drive head assembly for a syringe pump usable with a syringe having a barrel and a plunger, the plunger having a plunger flange, the drive head assembly being configured to drive the plunger along its axis when loaded in the syringe pump, the drive head assembly comprising:
a flange retaining mechanism for retaining the plunger flange to the drive head assembly, and a load measuring device for measuring compressive force on the plunger flange, the load measuring device being movable within the drive head assembly transverse to the axis;
wherein the load measuring device is arranged to receive a pushing force from a pushing component such that the load measuring device is moved within the drive head assembly until the pushing component abuts against the plunger flange or a datum that corresponds to the size of the plunger flange, such that a center of the load measuring device is aligned with the axis.

8. A drive head assembly according to claim 7, wherein the pushing component is located on the drive head assembly.

9. A drive head assembly according to claim 7, wherein the load measuring device is coupled to a transversely movable element having an engagement portion, and wherein the pushing component has at least a portion which is arranged to contact the engagement portion to move the transversely movable element.

10. A drive head assembly according to claim 7, wherein the flange retaining mechanism comprises at least one rotationally mounted arm arranged to engage with the plunger flange.

11. A drive head assembly according to claim 7, wherein the pushing component is part of the flange retaining mechanism.

12. A method of loading a syringe in a syringe pump according to claim 1, the syringe having a barrel with a barrel flange, and a plunger arranged to move within the barrel and having a plunger flange, the method comprising:
placing the barrel in a cradle of the syringe pump;
activating the flange retaining mechanism to retain the plunger flange; then,
activating the drive mechanism to push the plunger flange such that the barrel flange abuts against an engagement surface of the syringe pump.

13. A syringe pump usable with a syringe having a barrel and a plunger, the plunger having a plunger flange, the syringe pump comprising:
a barrel retaining mechanism for holding the barrel; and
a drive mechanism having a drive head assembly for driving the plunger along its axis, the drive head assembly comprising a flange retaining mechanism for retaining the plunger flange to the drive head assembly, and a load measuring device for measuring compressive force on the plunger flange, the load measuring device being movable within the drive head assembly transverse to the axis; and
a pushing component which is arranged to advance towards the load measuring device and to move the load measuring device within the drive head assembly until the pushing component abuts against the plunger flange or a datum that corresponds to the size of the plunger flange, such that a center of the load measuring device is aligned with the axis.

14. A syringe pump usable with a syringe having a barrel and a plunger, the plunger having a plunger flange, the syringe pump comprising:
a housing arranged to receive the syringe, the housing having a drive mechanism having a drive head assembly for driving the plunger along its axis;
the drive head assembly including a load measuring device for measuring compressive force on the plunger flange and a push button clamp arranged to clamp the plunger flange against the drive head assembly.

15. A syringe pump according to claim 14, wherein the drive assembly further including a flange bracket, with the push button clamp being spring loaded and arranged to apply a resilient bias to the plunger flange;
wherein the push button clamp and flange bracket define a bay arranged to receive and resiliently clamp the plunger flange.

16. A syringe pump according to claim 15, wherein the load measuring device is located centrally within the bay, the push button clamp and flange bracket arranged to clamp the plunger flange so as to align the load measuring device with a longitudinal axis of the syringe.

17. A syringe pump according to claim 14, wherein the recess includes a safety aperture, said safety aperture arranged to receive a safety projection from said syringe, such that the syringe pump is prevented from operation until said safety aperture has received said safety projection.

18. A syringe for use with a syringe pump, said syringe including a barrel, a safety projection and a plunger, the plunger having a plunger flange.

19. A syringe according to claim 18, wherein the safety projection is arranged to be inserted into a safety aperture of said syringe pump.
